(19) United States
(12) Patent Application Publication
(10) Pub. No.: US 2023/0389952 A1
(43) Pub. Date: Dec. 7, 2023

(54) ARRANGEMENT FOR A BLADED SURGICAL INSTRUMENT

(71) Applicant: CMR Surgical Limited, Cambridge (GB)

(72) Inventors: Ben Robert Chaplin, Cambridge (GB); David William Haydn Webster-Smith, Cambridge (GB)

(73) Assignee: CMR Surgical Limited, Cambridge, EN (GB)

(21) Appl. No.: 18/249,813

(22) PCT Filed: Oct. 22, 2021
(86) PCT No.: PCT/GB2021/052749

§ 371 Date: Apr. 20, 2023

(30) Foreign Application Priority Data
Oct. 23, 2020 (GB) 2016820.9

(51) Int. Cl. A61B 17/32; A61B 17/3201

(52) U.S. Cl. CPC A61B 17/320016; A61B 17/3201; A61B 2034/305

(57) ABSTRACT

A robotic surgical instrument comprising: an end effector with opposing first and second end effector elements, each end effector element comprising a first portion for enabling rotation of that end effector element about a respective joint and a second portion with a surface configured to interface with a corresponding surface of the opposing end effector element; and an articulation comprising: a first joint permitting rotation of the first end effector element about a first axis; a second joint permitting rotation of the second end effector element about a second axis; and a supporting body comprising opposing first and second tines within which the first portions of the first and second end effector elements are permitted to rotate; wherein the first portion of the first end effector element is proximal to the first tine, and the second portion of the first end effector element is proximal to the second tine.

### FIELD OF THE INVENTION

This invention relates to the configuration of the end effector elements of a bladed robotic surgical instrument.

### BACKGROUND OF THE INVENTION

Surgical robots are commonly being used to perform surgical procedures, due to the improvements in precision and sterility that they offer when compared to manual open or laparoscopy operations. A typical surgical robot comprises a base unit, a robot arm, and a surgical instrument. The robot arm is connected at its proximal end to the base unit, and at its distal end to the surgical instrument. The surgical instrument, at its distal end, comprises an end effector for penetrating the body of a patient at a port to reach a surgical site where it engages in a medical procedure.

One type of surgical instrument that is commonly used in combination with operational surgical robots is a bladed surgical instrument. Some bladed surgical instruments comprise a pair of opposing members, each member having an inner cutting surface configured to interface a corresponding cutting surface of the opposing member. During a surgical procedure, the instrument is able to lacerate and cut through thin layers of organic material situated between its opposing members. Advancements are continuously being made to improve on the existing configuration of bladed surgical instruments, in order to optimise the cutting accuracy and efficiency of these instruments, therefore enhancing their performance as they are used to perform robotic surgery.

There is a need for an improved bladed surgical instrument that provides the accuracy and efficiency improvements described above.

### SUMMARY OF THE INVENTION

According to a first aspect, there is provided a robotic surgical instrument comprising: an end effector with opposing first and second end effector elements, each end effector element comprising a first portion for enabling rotation of that end effector element about a respective joint and a second portion with a surface configured to interface with a corresponding surface of the opposing end effector element; and an articulation comprising: a first joint permitting rotation of the first end effector element about a first axis; a second joint permitting rotation of the second end effector element about a second axis; and a supporting body comprising opposing first and second tines within which the first portions of the first and second end effector elements are permitted to rotate; wherein the first portion of the first end effector element is proximal to the first tine, and the second portion of the first end effector element is proximal to the second tine.

The first portion of the second end effector element may be proximal to the second tine, and the second portion of the second end effector element may be proximal to the first tine.

The first end effector element and the second end effector element may be independently rotatable about the first and second axes respectively.

The first joint may be drivable by a first pair of driving elements.

The second joint may be drivable by a second pair of driving elements.

The end effector further may comprise a pair of biasing mechanisms, where each biasing mechanism acts as a spacer between the first or second end effector element and the first or second tine.

The first and second joints may be cylindrical pins, the first tine may comprise a recess for housing the first joint and the second tine may comprise a recess for housing the second joint.

An exterior surface of the first portion of the first end effector element may face a plane comprising the first tine, and an exterior surface of the second portion of the first end effector element may face a plane containing the second tine.

An exterior surface of the first portion of the second end effector element may face a plane containing the second tine, and an exterior surface of the second portion of the second end effector element may face a plane containing the first tine.

The exterior surfaces of the first and second portions of the first end effector element may oppose the surface of the first end effector element that is configured to interface a corresponding surface of the second end effector element.

The exterior surfaces of the first and second portions of the second end effector element may oppose the surface of the second end effector element that is configured to interface a corresponding surface of the first end effector element.

The exterior surface of the first portion of the first end effector element may be aligned with the exterior surface of the second portion of the second end effector element.

The exterior surface of the first portion of the second end effector element may be aligned with the exterior surface of the second portion of the first end effector element.

The robotic surgical instrument may further comprise a shaft, wherein the articulation connects the end effector to the shaft via the first and second joints.

The first tine and the second tine may extend in a direction that is parallel to the longitudinal axis of the shaft.

The first axis and the second axis may be the same axis.

The first and second end effector elements may be first and second jaws of an end effector.

The end effector element may be a pair of scissors.

The robotic surgical instrument may be configured to be connected to a surgical robot.

There may be provided a robotic surgical instrument comprising: an end effector with opposing first and second end effector elements, each end effector element comprising a first portion for enabling rotation of that end effector element about a respective joint and a second portion with a surface configured to interface with a corresponding surface of the opposing end effector element; and an articulation comprising: a first joint permitting rotation of the first end effector element about a first axis; a second joint permitting rotation of the second end effector element about a second axis; wherein the first joint is closer to the second portion of the second end effector element than it is to the second portion of the first end effector element.

The second joint may be closer to the second portion of the first end effector element than it is to the second portion of the second end effector element.

### BRIEF DESCRIPTION ON THE FIGURES

The present invention will now be described by way of example with reference to the accompanying drawings. In the drawings:

FIG. 1 illustrates a surgical robot;

FIG. 2 illustrates a first embodiment of a distal end of a surgical instrument;

FIG. 3 illustrates a common problem with the arrangement of the surgical instrument illustrated in FIG. 2;

FIGS. 4A, 4B and 4C illustrate a second embodiment of a distal end of a surgical instrument.

### DETAILED DESCRIPTION

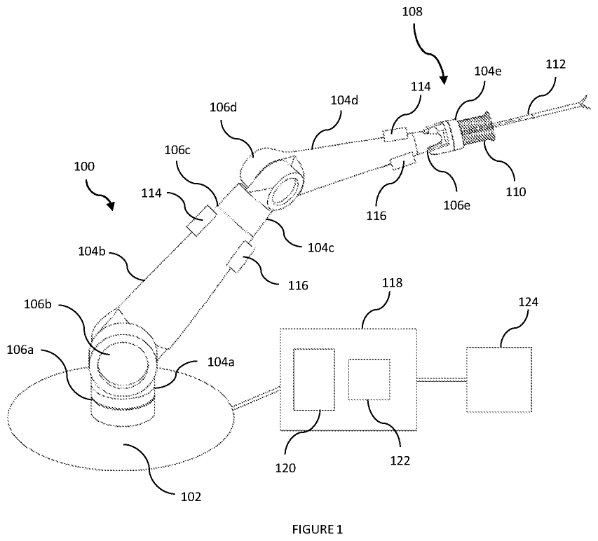

FIG. 1 illustrates a surgical robot having an arm 100 which extends from a base unit 102. The arm comprises a plurality of rigid limbs 104a-e which are coupled by a plurality of joints 106a-e. The joints 106a-e are configured to apply motion to the limbs. The limb that is closest to the base 102 is the most proximal limb 104a and is coupled to the base by a proximal joint 106a. The remaining limbs of the arm are each coupled in series by a joint of the plurality of joints 106b-e. A wrist 108 may comprise four individual revolute joints. The wrist 108 couples one limb (104d) to the most distal limb (104e) of the arm. The most distal limb 104e carries an attachment 110 for a surgical instrument 112. Each joint 106a-e of the arm 100 has one or more drive sources 114 which can be operated to cause rotational motion at the respective joint. Each drive source 114 is connected to its respective joint 106a-e by a drivetrain which transfers power from the drive source to the joint. In one example, the drive sources 114 are motors. The drive sources 114 may alternatively be hydraulic actuators, or any other suitable means. Each joint 106a-e further comprises one or more configuration and/or force sensors 116 which provides sensory information regarding the current configuration and/or force at that joint. In addition to configuration and/or force sensory data, the one or more sensors 116 may additionally provide information regarding sensed temperature, current or pressure (such as hydraulic pressure).

The arm terminates in an attachment for interfacing with the surgical instrument 112. The surgical instrument has a diameter less than 8 mm. The surgical instrument may have a 5 mm diameter. The surgical instrument may have a diameter which is less than 5 mm. The surgical instrument comprises an end effector for performing an operation. The end effector may take any suitable form. For example, the end effector may be smooth jaws, serrated jaws, a gripper, a pair of shears, a pair of scissors, a needle for suturing, a camera, a laser, a knife, a stapler, a cauteriser or a suctioner. The end effector may alternatively be an electrosurgical instrument such as a pair of monopolar scissors. The surgical instrument further comprises an instrument shaft and an articulation located between the instrument shaft and the end effector. The articulation comprises several joints which permit the end effector to move relative to the shaft of the instrument. The joints in the articulation are actuated by driving elements. These driving elements are secured at the other end of the instrument shaft to interface elements of the instrument interface. The driving elements are elongate elements that extend from the joints in the articulation through the shaft to the instrument interface. Each driving element can be flexed transverse to its longitudinal axis in the specified regions. For example, the driving elements may be cables.

The diameter of the surgical instrument may be the diameter of the profile of the articulation. The diameter of the profile of the articulation may match or be narrower than the diameter of the shaft. The attachment comprises a drive assembly for driving articulation of the instrument. Movable interface elements of the drive assembly interface mechanically engage corresponding movable interface elements of the instrument interface in order to transfer drive from the robot arm to the instrument. Thus, the robot arm transfers drive to the end effector as follows: movement of a drive assembly interface element moves an instrument interface element which moves a driving element which moves a joint of the articulation which moves the end effector.

Controllers for the drive sources 114 and sensors 116 are distributed within the robot arm 100. The controllers are connected via a communication bus to a control unit 118. The control unit 118 comprises a processor 120 and a memory 122. The memory 122 stores, in a non-transient way, software that is executable by the processor 120 to control the operation of the drive sources 114 to cause the arm 100 to operate. In particular, the software can control the processor 120 to cause the drive sources (for example via distributed controllers) to drive in dependence on inputs from the sensors 116 and from a surgeon command interface 124.

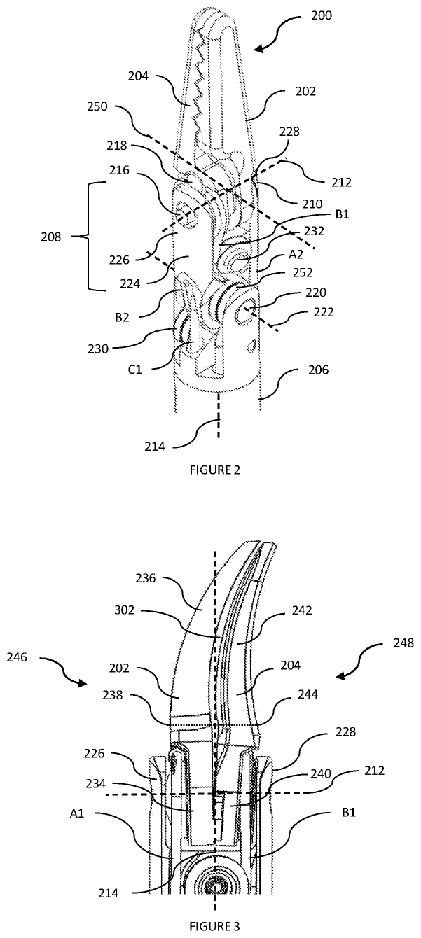

FIG. 2 illustrates a distal end of a surgical instrument for attachment to the arm of a surgical robot. The distal end of the surgical instrument is the end located furthest from the base unit of the surgical robot. The distal end of the surgical instrument comprises an end effector 200 with a pair of opposing end effector elements 202, 204. The end effector 200 is connected to the distal end of the shaft 206 by an articulation 208. The shaft is connected at its proximal end to an interface for attaching to a robot arm. The drive mechanism may comprise a drive source as described with reference to FIG. 1 above. Articulation 208 comprises joints which permit movement of the end effector 200 relative to the shaft 206. A first joint 210 permits the first end effector element 202 to rotate about a first axis. The first axis is transverse to the longitudinal axis of the shaft 214. A second joint 216 permits the second end effector element 204 to rotate about a second axis. The second axis is also transverse to the longitudinal axis of the shaft 214. The second axis may be parallel to the first axis. In one example, the first and second axes are the same axis, as is illustrated in FIG. 2 by reference numeral 212. However, it would be understood by the skilled person that, in alternative examples, the second axis is not the same as the first axis. For example, the second axis may be parallel to but offset from the first axis. The offset may be in a direction defined by the longitudinal axis of the shaft, or alternatively in a direction that is transverse to the longitudinal axis of the shaft. The offset may be in an alternative direction that is not defined with respect to the longitudinal axis of the shaft.

The first end effector element 202 and the second end effector element 204 may be independently rotatable about the first axis and the second axis respectively by the first and second joints. The end effector elements may be rotated in the same direction or different directions by the first and second joints. The first end effector element 202 may be rotated about the first axis, whilst the second end effector element 204 is not rotated about the second axis. The second end effector element 204 may be rotated about the second axis, whilst the first end effector element 202 is not rotated about the first axis. The shaft terminates at its distal end in the third joint 220. A third joint 220 permits the end effector 200 to rotate about a third axis 222. The third axis 222 is transverse to the first axis 212.

Each end effector element 202, 204 of the end effector 200 comprises a first portion and a second portion. The first portion of each end effector element is for enabling rotation of that end effector element about a respective joint. That is, the first portion of the first end effector element 202 is the portion of the end effector element that interacts with the first joint 210.

The first portion of the second end effector element 204 is the portion of the end effector element that interacts with the second joint 216. The second portion of each end effector element comprises a surface configured to interface with a corresponding surface of the opposing end effector element. That is, the second portion of the first end effector element 202 comprises a surface that is configured to contact a corresponding surface of the second portion of the second end effector element 204 when the first and second end effector elements are in a closed configuration. These contacting surfaces may otherwise be referred to as the inner surfaces of the first and second end effector elements, as they are located inside the end effector when they are interfaced. In one example, the end effector is a pair of scissors. In this example, the inner surfaces of the second portion of the first and second end effector elements are cutting surfaces. That is, the inner surfaces are configured to lacerate material situated between them when they moved towards the closed configuration.

In one example, the inner surfaces of the first and second end effector elements are configured to contact each other at their inner edges. That is, an inner edge of the first inner surface may be configured to contact an inner edge of the second inner surface. The first and second inner surfaces may be configured to contact each other at only their inner edges. The inner surfaces may be configured to contact each other at at least one location along their inner edges. This location may vary as the end effector elements are moved between an open and a closed configuration. The inner surfaces may be configured to contact each other at only one location along their inner edges. The location may move towards the distal tips of the end effector elements as they move towards a closed configuration. The distal tips of the end effector elements are the ends of the end effector elements that are furthest from the shaft.

The articulation 208 comprises a supporting body 224. At a first end, the supporting body 224 is connected to the end effector 200 by the first joint 210 and the second joint 216. At a second end opposing the first end, the supporting body 224 is connected to the shaft 206 by the third joint 220. The first joint 210 and second joint 216 permit the end effector elements 202, 204 to rotate relative to the supporting body 224 about the first and second axes 212.

The third joint 220 permits the supporting body 224 to rotate relative to the shaft 206 about the third axis 222. The supporting body comprises a first tine 226 and a second tine 228. The first and second tines extend from the base of the supporting body towards the distal end of the surgical instrument. The first tine 226 opposes the second tine 228. That is, the first tine 226 is located on an opposite side of the supporting body to the second tine 228. The first tine 226 and the second tine 228 are spaced apart, such that the first portions of the first and second end effector elements are permitted to rotate between the tines. The first tine and the second tine may extend towards the distal end of the surgical instrument in a direction that is parallel to the longitudinal axis of the shaft.

FIG. 2 illustrates the surgical instrument in a straight configuration. In this configuration, the end effector 200 is aligned with the shaft 206. That is, the longitudinal axis of the articulation and the longitudinal axis of the end effector are coincident with longitudinal axis 214 of the shaft. The first and second axes are both transverse to the longitudinal axis 214 of the shaft. Articulation of the first, second and third joints enables the end effector to take a range of attitudes relative to the shaft.

Each joint of the end effector is driven by a pair of driving elements. So, each joint is independently driven. The first joint 210 is driven by a first pair of driving elements A1, A2. The second joint 216 is driven by a second pair of driving elements B1, B2 (not visible). The third joint 212 is driven by a third pair of driving elements C1, C2 (not visible). At one point, driving elements of a pair of driving elements are secured to their corresponding joint. For example, the second pair of driving elements B1, B2 comprises a ball feature 218 which is secured to the second joint 216. The ball feature 218 may be otherwise referred to as a crimp. The first end effector element 202 may comprise an additional constraining feature for constraining the first pair of driving elements A1, A2. The first portion of the second end effector element 204 may also comprise an additional constraining feature for constraining the second pair of driving elements B1, B2. The constraining features ensure, that when the pair of driving elements is driven, the drive is transferred to motion of the joint about its axis.

The surgical instrument of FIG. 2 further comprises a pulley arrangement around which the first and second pairs of driving elements are constrained to move. The pulley arrangement comprises a first set of pulleys 252 rotatable about the third axis 222. That is, the first set of pulleys 252 rotate about the same axis as the third joint 220. The pulley arrangement further comprises at least a second set of pulleys 230 and a pair of redirecting pulleys 232. The pulley arrangement, and the routing of driving elements around this arrangement, may correspond to the arrangement described in PCT application no.: WO 2017/098279 A1.

A common problem with the arrangement of the end effector of FIG. 2 is illustrated in FIG. 3. This problem occurs most frequently for end effectors that comprise a pair of effector elements with opposing cutting surfaces. In FIG. 3, the end effector is a pair of scissors. Although the end effector elements illustrated in FIG. 3 are different from those illustrated in FIG. 2, corresponding reference numerals have been used to assist understanding the comparability between these two figures. That is, with the exception of the shape of the end effector elements, the surgical instruments of FIGS. 2 and 3 are the same.

As illustrated in FIG. 3, the first portion 234 of the first end effector element is proximal to the first tine 226. That is, the first portion 234 of the first end effector element is next to the first tine 226. The majority of the second portion 236 of the first end effector element is directly above the first portion 234 of the first end effector element. Thus, the second portion 236 of the first end effector element is also proximal to the first tine 226. That is, although the second portion 236 of the first end effector element is located above the first tine 226, it is adjacent to the first tine at a location 238 where it adjoins the first portion 234.

Correspondingly, the first portion 240 of the second end effector element is proximal to the second tine 228, and the second portion 242 of the second end effector is also proximal to the second tine 228. That is, although the second portion 242 of the second end effector element is located above the second tine 228, it is adjacent to the second tine at a location 244 where it adjoins the first portion 240. The majority of the second portion 242 of the second end effector element is directly above the first portion 240 of the second end effector element.

The end effector element can be divided into two halves, where a plane containing the longitudinal axis of the shaft 214 and extending transversely to the first and second axes 212 separates the first half 246 of the end effector from its second half 248. The first portion 234 of the first end effector element is located in the first half 246 of the end effector, and the first portion 240 of the second end effector element is located in the second half 248 of the end effector. The majority of the second portion 236 of the first end effector element is located in the first half 246 of the end effector, and the majority of the second portion 242 of the second end effector element is located in the second half 248 of the end effector. In other words, the majority of the first end effector element 202 as a whole is in the first half 246 of the end effector. The majority of the second end effector element 204 as a whole is in the second half 248 of the end effector.

To move the end effector from an open configuration to a closed configuration, a tensile force is applied to the driving elements of the first and second end effectors. That is, to move the first end effector element 202 towards a closed configuration a nominal tensile force is applied to driving element A1 of the first pair of driving elements. To move the second end effector element 204 towards a closed configuration a nominal tensile force is applied to driving element B1 of the second pair of driving elements.

The application of an increased tensile force to driving element A1 as compared driving element A2 results in rotation of the first end effector element 202 about the first axis via first joint 210. Correspondingly, the application of an increased tensile force to driving element B1 as compared to driving element B2 results in rotation of the second end effector element 204 about the second axis via second joint 216. As well as the rotation of the first and second end effector elements 202, 204 about the first and second axes respectively, the tensile force from the driving elements A1, B1 applies a further rotary force to the first and second end effector elements. That is, the tension in the driving elements A1, B1 also rotates the end effector elements about fourth and fifth axes 250 that are parallel to the third axis and intersect both the first and second joints and the longitudinal axis of the shaft 214. The first end effector element 202 is rotated about the fourth axis, and the second end effector element 204 is rotated about the fifth axis. The fifth axis may be parallel to the fourth axis. As illustrated in FIG. 2, in one example the fourth and fifth axes are the same axis 250. The first end effector element 202 is rotated away from the longitudinal axis of the shaft 214, and towards the first tine 226 about the fourth axis. Correspondingly, second end effector element is rotated away from the longitudinal axis of the shaft 214 and towards the second tine 228 about the fifth axis. The tension that is applied to the first and second end effector elements to rotate these elements away from the longitudinal axis of the shaft results in a net separation of the elements away from the plane that separates the first half of the end effector from its second half as the end effector elements are moved towards the closed configuration. This separation is annotated by reference 302 in FIG. 3.

It is also possible to apply an equal tensile force to both driving elements of a pair of driving elements, such as driving elements A1 and A2 for the first end effector element 202. In this scenario, the first end effector element 202 is in a static state and will not rotate about the first axis but is still experiencing a tensile force. In this static state, the tensile forces applied to the end effector element by the pair of driving elements also contribute to the rotary forces of that driving element about the fourth axis. A corresponding scenario may arise for second end effector element 204.

The negative effect of the separation between the first and second end effector elements is that it inhibits the ability of the end effector to lacerate thin materials, such as organic material to be penetrated during a surgical procedure. That is, if the separation between the end effector elements when they are in the closed configuration is greater than the thickness of the material to be cut, then that material cannot be cut. In an example where the inner surfaces of the end effector elements are configured to contact each other at only one location along their inner edges, a separation between the first and second end effector elements when they are in a closed configuration may result in a lack of contact at that one location. This lack of contact will significantly reduce the cutting performance of the end effector. Furthermore, the separation between the end effector elements leads to a reduction in the cutting force that is applied to these elements. This is because a proportion of the tensile force exerted by driving elements A1, B1 is converted into the rotary forces that drive the elements apart about the fourth and fifth axes. The reduction in the cutting force will further reduce the cutting performance of the end effector. Thus, the overall performance of the end effector is adversely affected by the separation resulting from the configuration of the end effector elements illustrated in FIGS. 2 and 3.

To mitigate the separation between the end effector elements described above, each end effector element may be provided with a biasing mechanism that acts to press the end effector element towards the longitudinal axis of the shaft 214. The end effector as a whole may comprise a pair of biasing mechanisms, and each biasing mechanism may be located between an end effector element and its corresponding tine. In one example, the biasing mechanism is a spring. Each spring applies a force to its respective end effector element that counteracts the force that rotates that end effector element away from the longitudinal axis of the shaft 214. That is, each spring applies a force that rotates its end effector element towards the longitudinal axis of the shaft 214. Each biasing mechanism may be a conical spring, which acts to separate the end effector element and its corresponding tine. In other words, the biasing mechanism acts as a spacer between an end effector element and its corresponding tine. Whilst the pair of biasing mechanisms may mitigate the separation between the inner surfaces of end effector elements 202, 204, they may be insufficient to eliminate this separation when used in isolation.

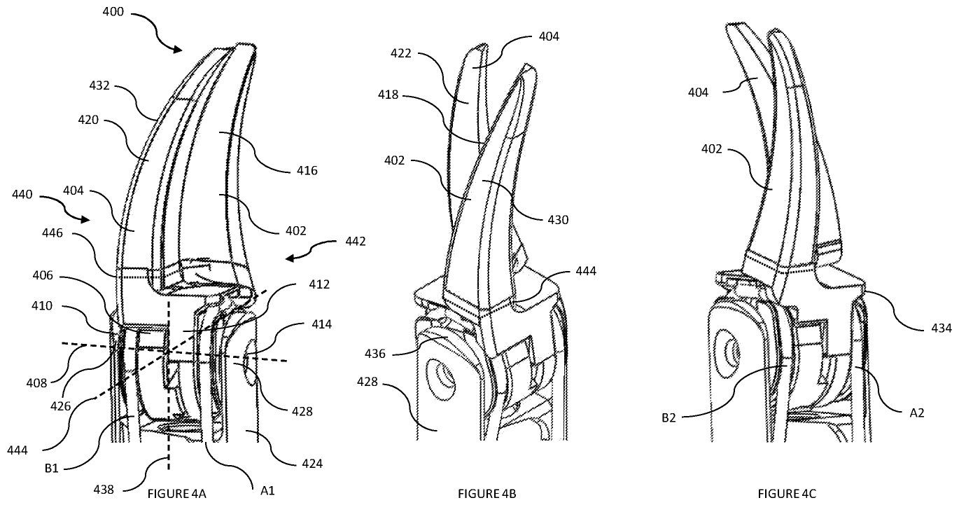

A second embodiment for the arrangement of a robotic surgical instrument which solves the problem illustrated in FIG. 3 is shown in FIGS. 4A-C. The arrangement of surgical instrument illustrated in FIG. 4 is the same as the arrangement illustrated in FIG. 2, but for the configuration of the end effector elements described below.

The surgical instrument comprises an end effector 400 with opposing first and second end effector elements. That is, the end effector comprises a first end effector element 402 and a second end effector element 404. Each end effector element comprises a surface configured to interface with a corresponding surface of the opposing end effector element. That is, the first end effector element 402 comprises a surface that is configured to contact a corresponding surface of the second end effector element 404 when the first and second end effector elements are in a closed configuration.

The surgical instrument illustrated in FIG. 4 also comprises an articulation as defined above with respect to FIG. 2. The articulation comprises first joint 410 that permits rotation of the first end effector element 402 about the first axis and second joint 414 permitting rotation of the second end effector element 404 about the second axis. The first axis and the second axis are illustrated in FIG. 4 as being the same axis 408. It would be appreciated that, in alternative examples, the first axis and the second axis may be distinct axes.

Each end effector element comprises a first portion for enabling rotation of that end effector element about a respective joint. That is, the first end effector element 402 comprises first portion 406. The first portion 406 of the first end effector element comprises a feature enabling the first end effector element to rotate about the first axis via the first joint 410. Correspondingly, the second end effector element 404 comprises first portion 412. The first portion 412 of the second end effector element comprises a feature enabling the second end effector element to rotate about the second axis via a second joint 414.

In one example, the first and second joints 410, 414 are cylindrical pins with an extruded length and a consistent cross-sectional area along that length. In this example, each of the first portions 406, 412 of the first and second end effector elements comprises a channel within which a corresponding joint is located, such that the channel can interact with the corresponding joint to enable rotation of the respective end effector element about the first and second axes 408. In an alternative example, the first and second joints 410, 414 are a single cylindrical pin.

Each end effector element further comprises a second portion with a surface configured to interface with a corresponding surface of the opposing end effector element. That is, the first end effector element 402 comprises a second portion 416 with an interfacing surface 418. The second end effector element 404 comprises a second portion 420 with a corresponding interfacing surface 422. The interfacing surface 418 of the first end effector element is configured to contact the corresponding interfacing surface 422 of the second end effector element when the first and second end effector elements are in a closed configuration. The interfacing surface 418 may contact the interfacing surface 422 an inner edge. That is, an inner edge of the interfacing surface 418 may be configured to contact the inner edge of the interfacing surface 422. The interfacing surfaces may be configured to contact each other at only their inner edges. The interfacing surfaces may be configured to contact each other at at least one location along their inner edges. This location may vary as the end effector elements are moved between an open and a closed configuration. The interfacing surfaces may be configured to contact each other at only one point location their inner edges. The location may move towards the distal tips of the end effector elements as they move towards a closed configuration, where the distal tips of the end effector elements are the ends of the end effector elements that are furthest from the shaft.

The interfacing surfaces may otherwise be referred to as the inner surfaces of the first and second end effector elements, as they are located inside the end effector when they are interfaced. As mentioned above, the end effector may be a pair of scissors, where the inner surfaces of the second portions of the first and second end effector elements are cutting surfaces.

The articulation further comprises a supporting body 424 corresponding to the supporting body 224 illustrated in FIG. 2. The supporting body 424 is connected to the end effector 400 by the first joint 410 and the second joint 414. The first joint 410 and second joint 414 permit the end effector elements 402, 404 to rotate relative to the supporting body 424 about the first and second axes 408. The supporting body comprises a first tine 426 and a second tine 428. The first and second tine extend from the base of the supporting body towards the distal end of the surgical instrument. The first tine 426 opposes the second tine 428. That is, the first tine 426 is located on an opposite side of the supporting body to the second tine 428. The first tine 426 and the second tine 428 are spaced apart, such that the first portions 406, 412 of the first and second end effector elements are permitted to rotate between the tines.

The arrangement of the end effector element illustrated in FIG. 4 is such that the first portion 406 of the first end effector element is proximal to the first tine 426, and the second portion 416 of the first end effector element is proximal to the second tine 428. Correspondingly, the first portion 412 of the second end effector element is proximal to the second tine 428, and the second portion 420 of the second end effector element is proximal to the first tine 426. The first tine 426 comprises a recess for housing the first joint 410. The second tine 428 comprises a recess for housing the second joint 414. Where the first and second joints are cylindrical pins, the recesses in the first and second tines may be channels that interact with a corresponding joint to enable rotation of a respective end effector element about the first and/or second axis.

Where the first portion 406 of the first end effector element is described as being proximal to the first tine 426, this means that the first portion is next to the first tine. In contrast to the example illustrated in FIG. 2, in FIG. 4 the end effector elements cross over so that the majority of the second portion 416 of the first end effector element is directly above the first portion 412 of the second end effector element. Thus, the second portion 416 of the first end effector element is proximal to the second tine 428. That is, although the second portion 416 of the first end effector element is located above the second tine 428, it is adjacent to the second tine at a location 446 where it adjoins the first portion 406.

Correspondingly, the first portion 412 of the second end effector element is proximal to the second tine 428, and the second portion 420 of the second end effector is proximal to the first tine 426. That is, although the second portion 420 of the second end effector element is located above the first tine 426, it is adjacent to the first tine at a location 446 where it adjoins the first portion 412. The majority of the second portion 420 of the second end effector element is directly above the first portion 406 of the first end effector element.

Because of the configuration described above, the end effector is arranged such that the first joint about which the first end effector element rotates is closer to the second portion of the second end effector element than it is to the second portion of the first end effector element. In other words, the first joint is closer to a part of the second portion of the second end effector element than it is to a respective part of the second portion of the first end effector element. For example, the first joint may be closer to the most proximal part of the second portion of the second end effector element than it is to the most proximal part of the second portion of the first end effector element.

Correspondingly, the second joint about which the second end effector element rotates is closer to the second portion of the first end effector element than it is to the second portion of the second end effector element. In other words, the second joint is closer to a part of the second portion of the first end effector element than it is to a respective part of the second portion of the second end effector element. For example, the second joint may be closer to the most proximal part of the second portion of the first end effector element than it is to the most proximal part of the second portion of the second end effector element.

The arrangement of the end effector in this way is such that, when the driving elements are pulled so as to bring the end effector elements together by rotating them about the first and second axes, the interfacing surfaces of the end effector elements are also brought together along the length of these axes.

The first joint 410 around which the first end effector element 402 is configured to rotate is drivable by a first pair of driving elements A1, A2. Correspondingly, the second joint 414 around which the second end effector element 404 is configured to rotate is drivable by a second pair of driving elements B1, B2. The first portion 406 of the first end effector element comprises a constraining feature for constraining the first pair of driving elements A1, A2. The constraining feature may resemble a pulley. The constraining feature may comprise a recess for housing the first pair of driving elements A1, A2, such that the driving elements are housed within the recess. The first pair of driving elements A1, A2 may be secured within the recess by a ball feature, such as the crimp illustrated in FIG. 2. The first portion 412 of the second end effector element comprises a corresponding constraining feature for constraining the second pair of driving elements B1, B2. The constraining features of the first and second end effector elements permit contact between the driving elements and their respective end effector element, such that tensile force can be transmitted from the driving elements to the end effector elements to permit rotation of the end effector elements about the first and second axes.

As described above, the second portions 416, 420 of the first and second end effector elements comprise inner, or interfacing surfaces, that are configured to contact each other when the end effector elements are in a closed configuration. That is, as described above, the interfacing surfaces are configured to contact each other at at least one location along their inner edges. The second portions of the first and second end effector elements further comprise exterior surfaces that oppose their corresponding interfacing surfaces. That is, the second portion of the first end effector element comprises an exterior surface 430 located on an opposite side of the first end effector element to the inner surface of the first end effector element. The second portion of the second end effector element comprises an exterior surface 432 located on an opposite side of the second end effector element to the inner surface of the second end effector element.

The first portions 406, 412 of the first and second end effector elements also comprise exterior surfaces. The first portion of the first end effector element comprises exterior surface 434, and the first portion of the second end effector element comprises exterior surface 436. The exterior surface 434 of the first portion of the first end effector element faces a plane containing the first tine 426. The exterior surface 434 of the first portion of the first end effector element may be orientated so that it is parallel to the first tine 426. The exterior surface 434 of the first portion of the first end effector element may be a planar surface. The first tine 426 may also comprise a planar surface. Correspondingly, exterior surface 436 of the first portion of the second end effector element faces a plane containing the second tine 424.

The exterior surface 430 of the second portion of the first end effector element may face a plane containing the second tine 428. That is, the exterior surface 430 of the second portion of the first end effector element faces a plane containing an opposing tine to the exterior surface 434 of the first portion of that end effector element. The exterior surface 432 of the second portion of the second end effector element may face a plane containing the first tine 426. In other words, the exterior surface 432 of the second portion of the first end effector element faces a plane containing an opposing tine to the exterior surface 436 of the first portion of that end effector.

The exterior surface of the first portion of the first end effector element is at least partially aligned with the exterior surface of the second portion of the second end effector element. That is, where the end effector elements are curved monopolar blades, at least the base of the exterior surface 432 is aligned with the exterior surface 434. At least the base of the second portion of the first end effector element is located above the first portion of the second end effector element. The base of the second portion of the first end effector element may abut the first portion of the second end effector element. Correspondingly, the exterior surface of the first portion of the second end effector element is at least partially aligned with the exterior surface of the second portion of the first end effector element. The base of the second portion of the second end effector element is be located above the first portion of the first end effector element. The base of the second portion of the second end effector element may abut the first portion of the first end effector element.

The robotic surgical instrument illustrated in FIG. 4 further comprises a shaft, corresponding to the shaft illustrated in FIG. 2. The longitudinal axis of the shaft is referenced in FIG. 4A by reference numeral 438. The articulation of the instrument acts to connect the end effector 400 to the shaft via the first and second joints 410, 414. At a second end opposing the first end, the supporting body 424 is connected to the shaft by a third joint (not illustrated). The third joint functions in the same way as third joint 220 illustrated in FIG. 2. The third joint permits the supporting body 424 to rotate relative to the shaft about a third axis. The first tine 426 and the second tine 428 may extend towards the distal end of the surgical instrument in a direction that is parallel to the longitudinal axis of the shaft 438. That is, the first tine 426 and the second tine 428 may be aligned with the longitudinal axis of the shaft 438.

As with the arrangement illustrated in FIG. 3, the end effector element of FIG. 4 can be divided into two halves, where a plane containing the longitudinal axis of the shaft 438 and extending transversely to the first and second axes 408 separates the first half 440 from the second half 442. The first portion 406 of the first end effector element is located in the first half 440 of the end effector, and the first portion 412 of the second end effector element is located in the second half 442 of the end effector. The majority of the second portion 416 of the first end effector element is located in the second half 442 of the end effector, and the majority of the second portion 420 of the second end effector element is located in the first half 440 of the end effector. That is, the majority of the second portion 416 of the first end effector element is located in a different half of the end effector to the first portion 406 of that end effector element. The majority of the second portion 420 of the second end effector element is located in a different half of the end effector to the first portion 412 of that end effector element.

In the embodiments of the end effector illustrated in FIGS. 4A-C, the end effector elements are illustrated as a pair of curved monopolar scissors. As viewed from a plane containing both the first and second axes 408 and the longitudinal axis of the shaft 438, the curved surfaces of the monopolar scissors do not extend linearly towards the distal end of the surgical instrument. Rather, the curved surface of the second end effector element 422 intersects and crosses over the longitudinal axis of the shaft. Thus, whilst the majority of the second portion 420 of the second end effector element is located in the first half of the end effector 440, it will be appreciated that part of the second portion is nevertheless located within the second half 442 of the end effector. In an alternative example, the majority of the second portion 416 of the first end effector element may be located in the second half of the end effector 442, but part of the second portion of the first end effector element may nevertheless be located within the first half 440 of the end effector. In a further example, the end effector elements may comprise straight cutting surfaces. In this example, the end effector elements will, in fact, extend linearly towards the distal end of the surgical instrument. This linear extension is in a direction parallel to the longitudinal axis of the shaft 438. Thus, in this example, the interfacing surfaces of the end effector elements will not intersect or cross over the longitudinal axis of the shaft. Therefore, the whole of the second portion 420 of the second end effector element will be comprised within the first half of the end effector. Correspondingly, the whole of the second portion 416 of the first end effector element will be comprised within the second half 442 of the end effector.

The arrangement of the robotic surgical instrument illustrated in FIG. 4 is advantageous as it uses the moments about fourth and fifth axes parallel to the third axis (as referenced above) to rotate the end effector elements towards each other about this axis as they move towards the closed configuration. The fourth and fifth axes intersect both the longitudinal axis of the shaft 438 and the first and second joints. It is illustrated by reference numeral 444 in FIG. 4A. This opposes the effect of the arrangement illustrated in FIG. 2, in which the elements are rotated away from each other about the fourth and fifth axes. In other words, tensioning of the driving elements A1, B1 not only rotates the end effector elements about the first and second axes 408, but also provides an additional biasing force to rotate the elements together about the fourth and fifth axes. This assists in guiding the end effector elements together as they are moved towards the closed configuration. Thus, the arrangement of the end effector not only reduces the separation between the opposing elements in their closed configuration, but also contributes to and increases the net cutting force. Thus, the overall efficiency of the end effector is enhanced, and its cutting performance improved. The end effector may also comprise a pair of biasing mechanisms as described above with respect to FIG. 2, to further reduce the separation between opposing end effector elements.

The robot described herein could be for purposes other than surgery. For example, the robot could be used to inspect a manufactured article such as a car engine, and the instrument could be a tool for operating inside the engine.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. A robotic surgical instrument comprising:
an end effector with opposing first and second end effector elements, each end effector element comprising a first portion for enabling rotation of that end effector element about a respective joint and a second portion with a surface configured to interface with a corresponding surface of the opposing end effector element; and
an articulation comprising:
a first joint permitting rotation of the first end effector element about a first axis, the first joint being drivable by a first pair of driving elements;
a second joint permitting rotation of the second end effector element about a second axis, the second joint being drivable independently from the first joint by a second pair of driving elements; and
a supporting body comprising opposing first and second tines within which the first portions of the first and second end effector elements are permitted to rotate;
wherein the first portion of the first end effector element is proximal to the first tine, and the second portion of the first end effector element is proximal to the second tine.

2. A robotic surgical instrument as claimed in claim 1, wherein the first portion of the second end effector element is proximal to the second tine, and the second portion of the second end effector element is proximal to the first tine.

3. A robotic surgical instrument as claimed in claim 1, wherein the first end effector element and the second end effector element are independently rotatable about the first and second axes respectively.

4. A robotic surgical instrument as claimed in claim 1, wherein the end effector further comprises a pair of biasing mechanisms, where each biasing mechanism acts as a spacer between the first or second end effector element and the first or second tine.

5. A robotic surgical instrument as claimed in claim 1, wherein the first and second joints are cylindrical pins, and wherein the first tine comprises a recess for housing the first joint and the second tine comprises a recess for housing the second joint.

6. A robotic surgical instrument as claimed in claim 1, wherein an exterior surface of the first portion of the first end effector element faces a plane comprising the first tine, and an exterior surface of the second portion of the first end effector element faces a plane containing the second tine.

7. A robotic surgical instrument as claimed in claim 6, wherein an exterior surface of the first portion of the second end effector element faces a plane containing the second tine, and an exterior surface of the second portion of the second end effector element faces a plane containing the first tine.

8. A robotic surgical instrument as claimed in claim 6, wherein the exterior surfaces of the first and second portions of the first end effector element oppose the surface of the first end effector element that is configured to interface a corresponding surface of the second end effector element.

9. A robotic surgical instrument as claimed in claim 7, wherein the exterior surfaces of the first and second portions of the second end effector element oppose the surface of the second end effector element that is configured to interface a corresponding surface of the first end effector element.

10. A robotic surgical instrument as claimed in claim 7, wherein the exterior surface of the first portion of the first end effector element is aligned with the exterior surface of the second portion of the second end effector element.

11. A robotic surgical instrument as claimed in claim 7, wherein the exterior surface of the first portion of the second end effector element is aligned with the exterior surface of the second portion of the first end effector element.

12. A robotic surgical instrument as claimed in claim 1, further comprising a shaft, wherein the articulation connects the end effector to the shaft via the first and second joints.

13. A robotic surgical instrument as claimed claim 12, wherein the first tine and the second tine extend in a direction that is parallel to the longitudinal axis of the shaft.

14. A robotic surgical instrument as claimed in claim 1, wherein the first axis and the second axis are the same axis.

15. A robotic surgical instrument as claimed in claim 1, wherein the first and second end effector elements are first and second jaws of an end effector.

16. A robotic surgical instrument as claimed in claim 1, wherein the end effector element is a pair of scissors.

17. A robotic surgical instrument as claimed in claim 1, wherein the robotic surgical instrument is configured to be connected to a surgical robot.
